(19) United States
(12) Patent Application Publication
(10) Pub. No.: US 2014/0135721 A1
(43) Pub. Date: May 15, 2014

(54) WOUND CARE ARTICLE HAVING A PATTERN OF INCISIONS AND/OR PUNCHINGS

(71) Applicant: Birgit Riesinger, Munster (DE)

(72) Inventor: Birgit Riesinger, Munster (DE)

(21) Appl. No.: 14/062,706

(22) Filed: Oct. 24, 2013

Related U.S. Application Data
(63) Continuation of application No. PCT/EP2012/057648, filed on Apr. 26, 2012.

(30) Foreign Application Priority Data
Apr. 26, 2011 (DE) 10 2011 002 268.6

(51) Int. Cl. A61F 13/20; A61F 13/00

(52) U.S. Cl. CPC A61F 13/2002; A61F 13/00068; A61F 13/00063
USPC 604/360; 604/369

(57) ABSTRACT

The invention relates to a wound care article, comprising a planar absorption body made of a nonwoven material or air-laid material containing superabsorbent polymers, wherein said planar absorption body has a pattern of incisions and/or punchings.

### CROSS-REFERENCE TO RELATED APPLICATIONS

The present application claims priority from and is a continuation from PCT Application No. PCT/EP2012/057648, filed Apr. 16, 2012; which claims priority from German Patent Application No. DE 102011002268.6, filed Apr. 26, 2011, all of which herein are incorporated by reference in their entireties.

### BACKGROUND

The present invention relates to a wound care article having a pattern of incisions and/or punchings according to the preamble of claim 1.

The prior art, for example, DE10059439 to the applicant of the present application, discloses wound care articles having a wound exudate absorption body. In this case, the latter has a substantially planar material portion of absorption material consisting of an absorbent nonwoven with superabsorbent particles dispersed therein.

The absorbent body described therein is composed of multiple layers, the layers being connected in a planar manner by means of so-called “bonding points”. The bonding points are created by thermal pressing with a plunger-like matrix. A wound care article produced with such bonding points has certain stiffness across its area and is not very flexible. In addition, under certain circumstances, the depressions created by the bonding method have welded surfaces on their walls, since the bonding method operates with heat, among other things. This may cause a sealing of the areas concerned and complicate the entry of liquids into the absorption material, which in turn affects the overall absorption of liquid.

There are other approaches that work without said bonding points. However, the products produced by these methods have a certain degree of stiffness and can also tend to complicate the entry of liquid into the absorption material.

### SUMMARY OF THE INVENTION

The object of the present invention is to provide a wound care article that does not have the disadvantages mentioned above. This object is achieved by the features of the present claim set. The dependent claims provide preferred embodiments. It should be noted that the ranges stated are to be understood throughout including the respective limit values.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

The present invention is illustrated in more detail by the figures shown and discussed hereinafter. It should be noted that the figures are only descriptive in nature and are not intended to limit the invention in any way.

FIG. 1a shows a wound care article 10 in plain view, comprising an envelope 11 having a seam 12, and a planar absorption body 13 made of a nonwoven or air-laid material containing superabsorbent polymers. The envelope forms an expansion space 14, so as to ensure that the absorption body can increase in volume upon liquid absorption and is not restricted by the envelope. The planar absorption body 13 has a pattern of L-shaped incisions 15 which are introduced in the absorption body by means of a correspondingly configured punching tool, and in particular increase the conformability of the originally relatively stiff absorption body. In this way, a wound care article is achieved that gently clings to the wound relief, is perceived by patients as very soft and comfortable and can fully develop its wound exudate absorbing function through close contact with the wound.

FIG. 1b shows examples of other shapes that can be adopted by said incisions. In all cases of FIG. 1, these incisions facilitate the entry of liquid into the absorption body and therefore accelerate the absorption of exudate. Due to the specific shape of the incisions, the material in this area can swell locally so as to ensure retention of absorbed liquid in place, and that no lateral distribution takes place.

Furthermore, in some cases, superabsorbent particles may leak and result in remoistening of the wound. This can be particularly advantageous when the absorption body is laminated with the cover layer which is to provide a dry surface of the absorption body. In some cases this can lead to local drying processes in the wound, which are remedied by said leakage of said superabsorbent particles.

FIG. 2a shows another wound care article 20 in plan view, comprising an envelope 21 having a planar absorption body 23 made of nonwoven material or air-laid material containing superabsorbent polymers. The planar absorption body 23 has a line-shaped pattern of punchings 25, which are introduced in the absorption body by means of a correspondingly configured punching tool. In this way, a folding line is defined along which the wound care article can be folded, for example, to insert into a deep wound. Furthermore, the punchings resting in particular at the wound bed facilitate the entry of liquid into the wound care product and thus improve the active absorption of exudate. FIG. 2b shows said wound care article 20 in the folded position in a deep wound.

FIG. 3a shows another wound care article 30 in plain view, comprising an envelope 31 and a planar absorption body 33 made of nonwoven material or air-laid material containing superabsorbent polymers. The planar absorption body 33 has three line-shaped patterns of punchings 35, which are introduced in the absorption body by means of a correspondingly configured punching tool. In this way, three folding lines are defined along which the wound care article can be folded, for example, to adapt it to the relief of a wound. Furthermore, the punchings resting in particular at the wound bed facilitate the entry of liquid into the wound care product and thus improve the active absorption of exudate. FIG. 3b shows said wound care article 30 in a folded position adapted to the relief of a wound.

FIG. 4 shows a further wound care article 40 in plain view, comprising an envelope 41 and a planar absorption body 43 made of nonwoven material or air-laid material containing superabsorbent polymers. The planar absorption body 43 has a planar pattern of punchings holes 45, which are introduced in the absorption body by means of a correspondingly configured punching tool. This facilitates the entry of liquid into the wound care article. Furthermore, the conformability of the originally relatively stiff absorption body is increased, so that a wound care article is achieved that gently clings to the wound relief, is perceived by patients as very soft and comfortable and can fully develop its wound exudate absorbing function through close contact with the wound.

FIG. 5 shows further embodiments of the wound care article according to the invention, wherein the punchings and/or incisions that are partly implemented together in a wound care article, increase the softness, the conformability and/or the comfort of the wound care article, facilitate the entry of liquid into the wound care article and/or define fold lines along which the wound care article can be folded.

FIG. 6 shows how superabsorbent particles can leak from the incisions (FIG. 6a) and/or punchings (FIG. 6b) and can provide for remoistening of the wound. This can be particularly advantageous when the absorption body is laminated with a cover layer which is to provide a dry surface of the absorption body. In some cases this can lead to local drying processes in the wound, which are remedied by said leakage of superabsorbent particles. In some instances, due to the specific shape of the incisions (FIG. 6a), the material in this area can swell locally so as to ensure retention of absorbed liquid in place, and that no lateral distribution takes place.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

According to the invention, therefore, it is provided a wound care article comprising a planar absorption body made of a nonwoven material or air-laid material containing superabsorbent polymers, having a pattern of incisions and/or punchings.

The term “nonwoven” means a textile fabric of individual fibers, which, as opposed to woven fabrics, crocheted fabrics and knitted fabrics, is not produced from yarns. Nonwoven fabrics retain their structural integrity usually by the individual fibers adhering to one another. They are also called “nonwovens”, and are produced by fulling the fibers, for example. The term “air-laid” refers to a specific nonwoven fabric made of wood pulp and polyolefin fibers optionally having embedded superabsorbent polymers.

Preferably, there is further provided that the wound care article contains at least partially superabsorbent polymers. Superabsorbent polymers (SAP) are plastics that are able to absorb liquids many times their own weight—up to 1000 times. Chemically, it is a copolymer of acrylic acid (propenoic acid, C₃H₄O₂) and sodium acrylate (sodium salt of acrylic acid, NaC₃H₃O₂), wherein the ratio of the two monomers can vary.

In addition, a so-called core cross-linker (CXL) is added to the monomer solution, which connects the long chain polymer molecules formed in places with one another by chemical bonds (“crosslinking”). These bridges render the polymer water-insoluble. Upon entry of water or aqueous salt solutions into the polymer particles it swells and tightens this network at the molecular level, so that the water cannot escape without help.

In the wound care article according to the invention the superabsorbent polymers may be present in the form of granules, a powder, a bulk, a pellet, a foam, in the form of fibers, a fiber knitted fabric, laid webs, or nonwoven and/or a fiber wadding.

Preferably, the wound care article may have a substantially planar absorption body made of absorption material, which consists of an absorbent nonwoven having superabsorbent polymers dispersed therein. They may be present in the form of granules, a powder, a bulk, a pellet, a foam, in the form of fibers, a fiber knitted fabric, laid webs, or nonwoven and/or a fiber wadding.

In this context, the absorption body has at least one material which is selected from the group consisting of a mat, in particular an air-laid made of said yarns or fibers made of superabsorbent polymers with incorporated superabsorbent polymers, and/or a loose fill of superabsorbent polymers. Preferably, said air-laid mat may have a substantially planar material portion made of absorption material which consists of an absorbent nonwoven made of said fibers having superabsorbent polymers dispersed therein.

This absorption body can correspond to the absorbent padding which is contained in a wound dressing of the applicant of the present invention, as disclosed, for example, in WO 2003/094813, WO 2007/051599 and WO 2001/52780, all incorporated by reference in their in their entireties, and distributed under the trade name “sorbion sachet”. The disclosure of said specifications is incorporated herein by reference in its entirety.

In another embodiment the absorption body can also form a core, having—optionally flaky—fibers or yarns made of superabsorbent polymers and superabsorbent polymers in the form of granules, wherein the granules are glued or welded to the fibers and yarns, respectively, in several heights, and the granules are distributed over more than 50% of the total height of the structure of at least a portion of the core, wherein blended sections of granules and fibers are present. In this context, preferably, the proportion by weight of the superabsorbent polymers can be in the range of 10-25% by weight. Similar structures are known from conventional incontinence materials and known for their cushioning properties such as sanitary napkins Around said core, there may be disposed an envelope, which in sections is arranged in an overlapping fashion, and which is covered by an adhesive seam or wherein the adhesive seam is part thereof.

Also, within the envelope there may be provided a portion of a hydrophobic and/or water-repellent or water-impermeable material which acts as barrier layer or clothing protection.

In another embodiment the absorption body may also contain at least one planar layer comprising fibers or yarns made of superabsorbent polymers, to which superabsorbent polymer in the form of granules are glued. In a preferred embodiment this results in a structure of the body having at least three layers, wherein two cover layers are surrounding a layer comprising superabsorbent polymers.

In this case, in the plane there are no blends of fibers and superabsorbent polymers; but only areas of materials fixed adjacent to one another. Here, in a preferred embodiment, the optionally provided several layers may be compressed physically by rolling, pressing, calendaring or similar methods.

Moreover, the body may have repeated patterns or textures such as, for example, a checked pattern, a punching pattern or the like.

The term “incision”, as used herein, refers to a cut into the absorption body, wherein no material has been removed. The term “punching”, as used herein, refers to a cut into the absorption body, wherein material has been removed.

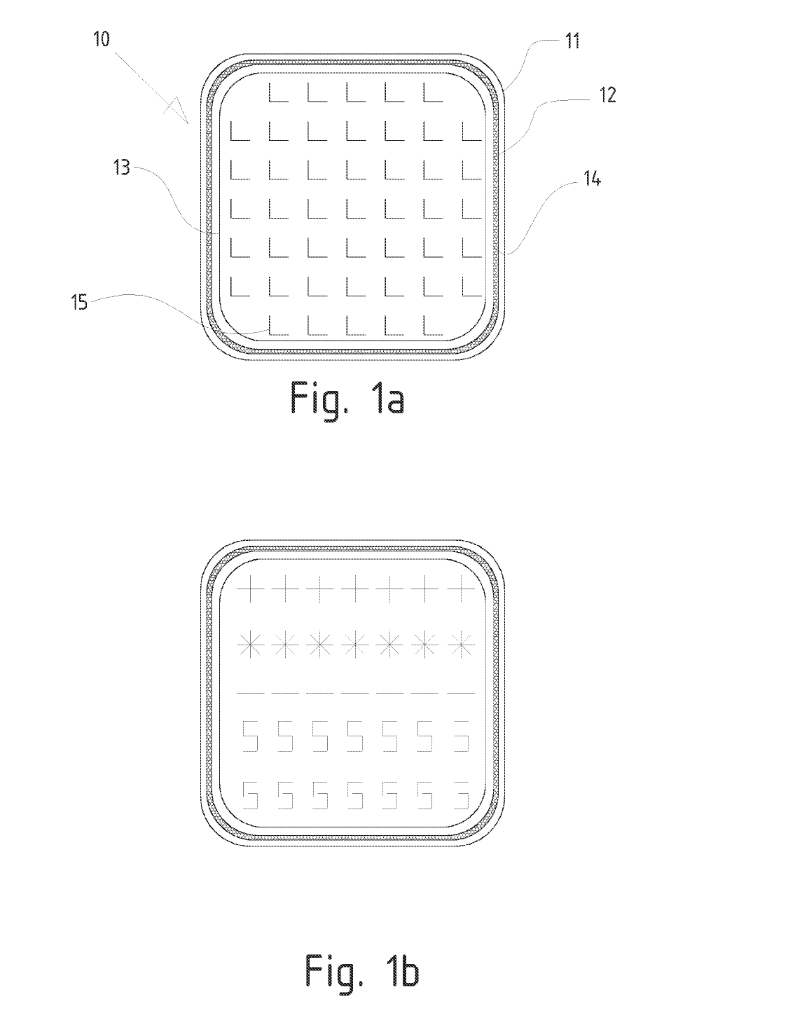

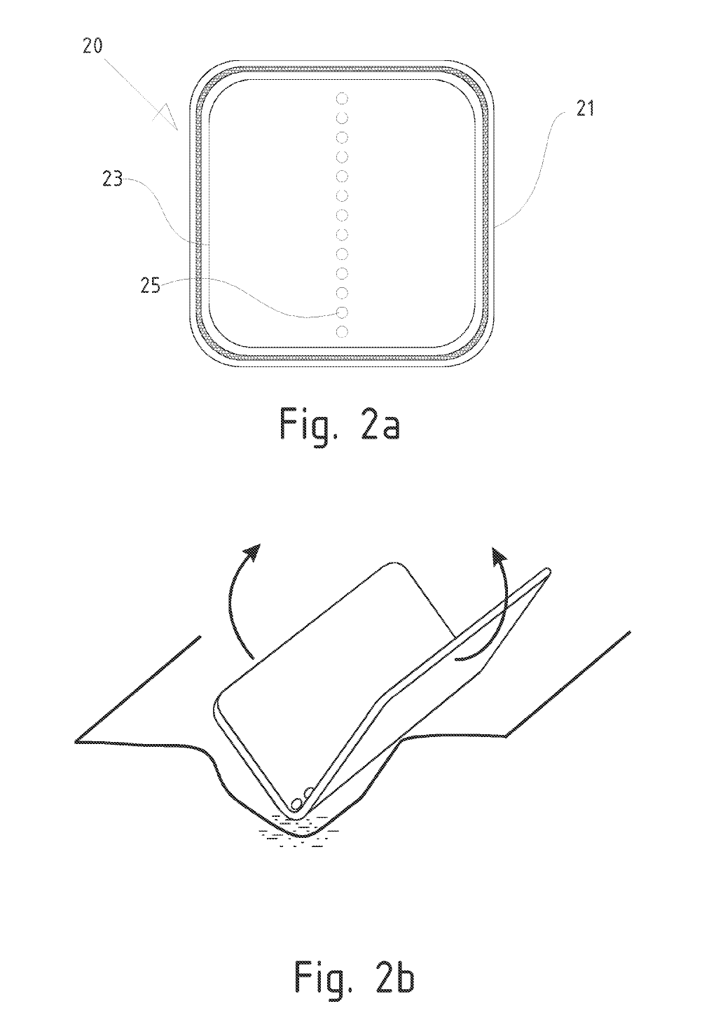

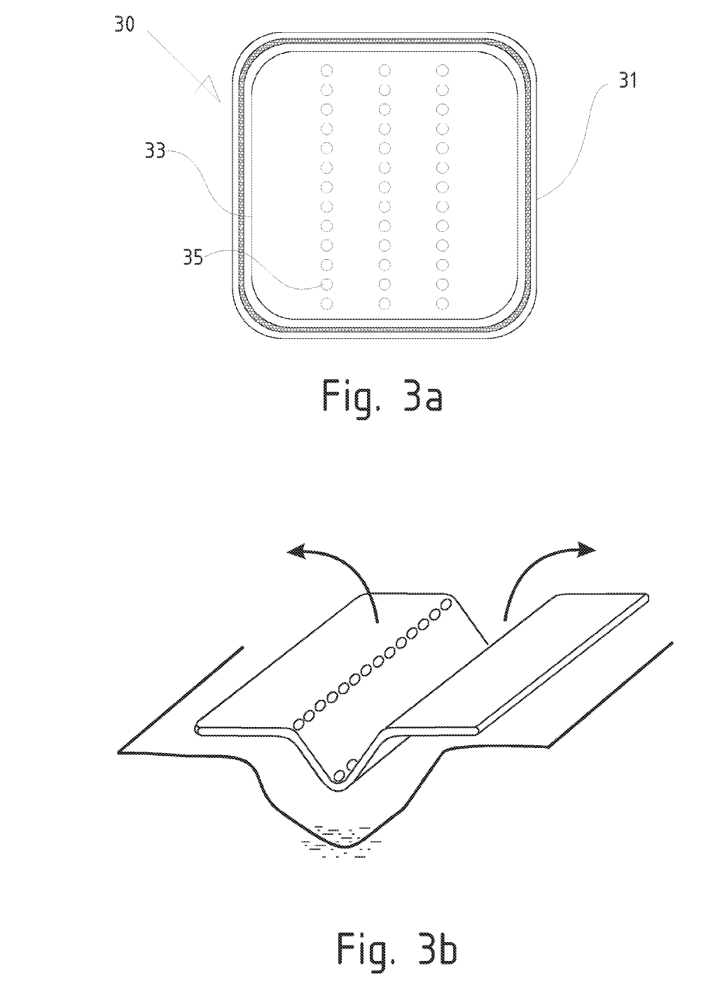

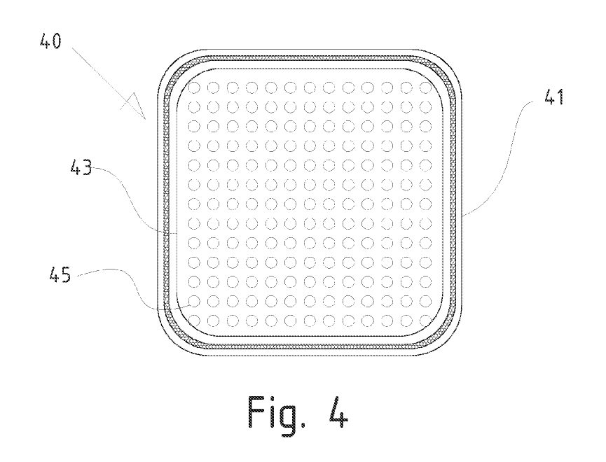

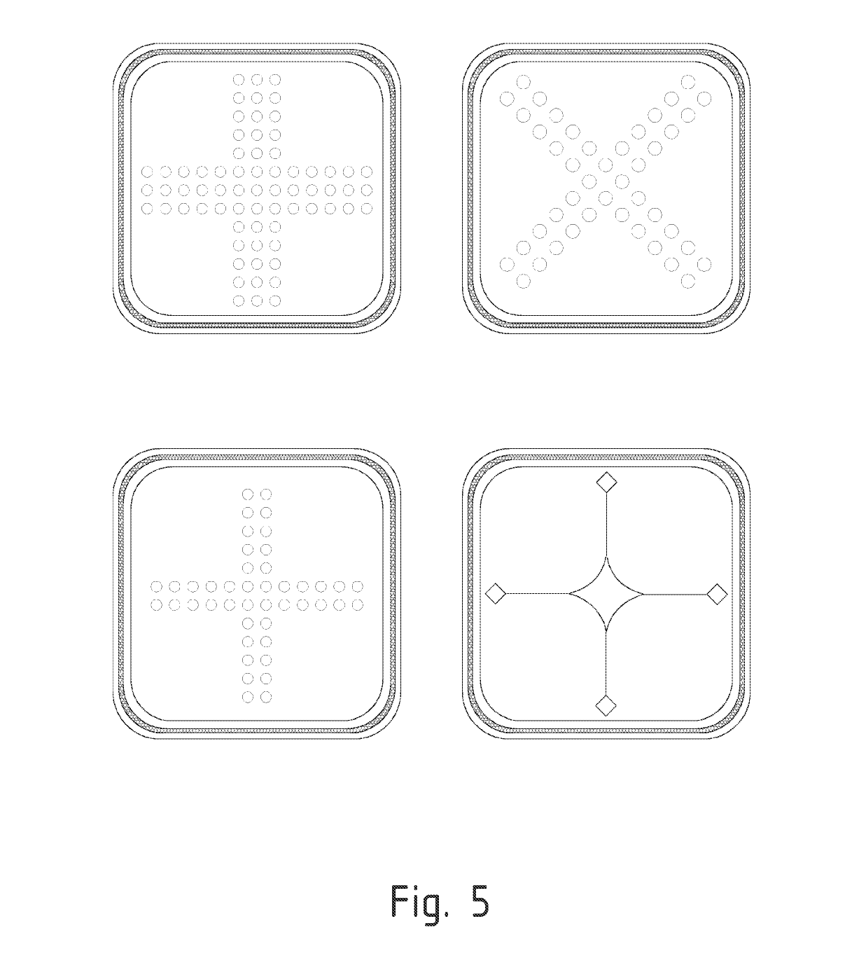

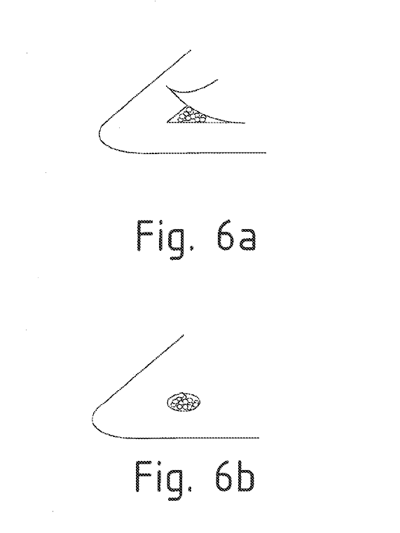

Incisions in the context of the present invention are preferably cuts into the material without substantially removing material. Said incisions may be slit-shaped or angle-shaped (L-shaped), but also cross-shaped or star-shaped. Other preferred shapes of said incisions are shown in FIG. 1b.

Punchings in the context of the present invention are characterized by a substantial removal of material and are preferably circular (“hole punching”), diamond-shaped, or polygonal. Of course, combinations of incisions and punchings are conceivable in a wound care article.

Preferably, both the incisions and the punchings are formed to run through all layers of the absorption body. Alternatively, it is also provided that they only run through a few layers of the absorption body.

It is important here that the shape of the incisions and/or punchings, and their depth can be specified by the respective punching or incision tools.

It is preferably provided that the incisions and/or punchings are formed and/or arranged in such a way so that they enhance the softness, conformability and/or the wearing comfort of the wound care article. Similarly, it is preferably provided that the incisions and/or punchings are formed and/or arranged in such a way so that they facilitate the entry of liquids into the wound care article. This can be particularly advantageous when the absorption body is laminated with a cover layer which should provide for a dry surface of the absorption body. In some cases this cover layer can hinder the entry of liquid into the absorption body. Providing incisions and/or punchings according to the invention will counteract this hindrance.

Furthermore, in some cases, superabsorbent particles may leak from the incisions and/or punchings and result in remoistening of the wound. This can be particularly advantageous when the absorption body is laminated with the cover layer mentioned above which is to provide a dry surface of the absorption body. In some cases this can lead to local drying processes in the wound, which are remedied by said leakage of superabsorbent particles.

Furthermore, it is preferred that the incisions and/or punching are formed and/or arranged in such a way so that they define folding lines, along which the wound care article can be folded.

Furthermore, the punchings create a kind of “internal expansion space” which is available for an increase in volume of the absorption material as a result of liquid absorption and thus is promoting the latter.

In a preferred embodiment, it is provided that the wound care article comprises an at least partially liquid-permeable envelope. The latter surrounds the absorption body, forms a barrier against solid excretions and allows the passage of other leaking substances to the absorption body arranged within the envelope. The envelope is preferably at least partially closed by a seam. This may be, for example, be an adhesive seam, a stitched seam or a welded seam, such as an ultrasonic weld.

In a further preferred embodiment, it is provided that the planar absorption body, in top view onto its planar side, has an area (F1) which in the non-wetted state is 3% to 75% smaller than the area (F2) of the flattened envelope.

In this way, the wound care article forms an expansion space, which ensures that the absorption body can increase in volume when it absorbs liquid and is not restricted by the envelope.

Particularly preferably, it provided that, in plain view on its planar side, the envelope has an encircling upper level that is protruding from the seam and the absorption body is free of hard, sharp edges and corners. Particularly preferably, in plain view, the corners of the envelope are very rounded.

Preferably, the pores or meshes of the envelope are configured such that they are smaller than the superabsorbent particles. This is of particular importance because superabsorbent particles may trickle out from the absorption body through the incisions or punchings and therefore must be retained by the envelope.

Although the size of the superabsorbent particles is subject to certain stochastic variations, it can be provided that the pores or meshes of the envelope are preferably smaller than the mean diameter of said superabsorbent polymer particles, more preferably smaller than the mean diameter of the single or multiple standard deviations of said superabsorbent particles.

Preferably, the pores may have a size of 0.05 mm to 1.0 mm, preferably 0.20 mm to 0.50 mm, for example. It may further be preferred that the pores or meshes bounded by the filament or fiber sections which are approximately arc-shaped in a section through the envelope with their arc apices pointing outwards.

In a further preferred embodiment it is provided, that the absorption body is fixed in the case. Alternatively, however, it may also be arranged to move freely in the envelope.

It is particularly preferred that the absorption body in the wound care article according to the invention has a grammage or an area weight of ≧100 g/m² and ≦600 g/m², more preferably ≧125 g/m² to ≦500 g/m² and particularly preferably of ≧160 g/m² to ≦220 g/m². It is also preferred that the wound care article according to the invention has a width of between 20 and 140 cm, both inclusive, and a length between 30 cm and 250 cm, both inclusive. Particularly preferred dimensions are 7.5×7.5 cm, 10×10 cm, 10×20 cm and 20×20 cm.

In a further preferred embodiment it is provided, that the absorption body has a content of 40% by weight of superabsorbent polymers. Particularly preferably, the weight proportion of the superabsorbent polymers is ≧45, 50, 55, 60, 65 or 70% by weight. Wound care articles with such high weight proportions of superabsorbent polymers have a particularly high absorption capacity for liquids and can replace marketed vacuum wound care systems.

The envelope is preferably formed of woven or nonwoven composite synthetic fibers such as polypropylene or polyethylene fibers, but also cotton, silk or rayon. Preferably, the envelope consists of a woven or a nonwoven fabric having an area-specific weight of at least 20 g/m².

Preferably, the envelope is consists of hydrophobic material or the envelope material is rendered hydrophobic. The hydrophobic properties of the envelope prevent it from adhering to the wound surface and contribute to the exudate-particles being able to seep into the interior of the shell faster.

In a further preferred embodiment it is provided that the envelope comprises at least partially an elastic material. This may be fibers made of Lycra or elastane, for example. This also ensures that the material section upon liquid absorption may increase in its volume and is not restricted by the envelope. In this case, the expansion space described above is optionally dispensed with.

Wound dressings of this type are disclosed in WO 2003/094813, WO 2007/051599, and WO 2001/52780 to the applicant of the present invention, for example. The disclosure of said specifications is herein incorporated by reference in its entirety.

The material of the envelope may be structured in such a way so that the envelope has a rough inner surface and a smooth outer surface. Preferably, the rough inner surface of the envelope is formed by funnel-shaped perforations, which taper towards the inner surface and end in a free opening edge (“projection”).

This rough inner surface counteracts the displacement of the contents of the envelope, so that fixing with adhesion points can be omitted. Accordingly, the smooth outer surface of the envelope material may be formed by curved material sections extending between the perforations. As opposed to a material that is planar on both sides, such envelope material can be termed “three-dimensional”, and is known from DE102006017194 to the applicant of the present application, the disclosure of which is herein incorporated by reference in its entirety.

Particularly preferably, it is provided that said three-dimensional envelope material is laminated to the aforementioned nonwoven polypropylene. As a consequence, such embodiment has improved liquid absorption properties.

There may be provided also, that the wound care article on its side facing away from the wound comprises a liquid-proof clothing protection (“back sheet”). Furthermore, it can be provided that the envelope on the side facing the wound is equipped with a physiologically acceptable adhesive (such as a hydrocolloid adhesive).

In a further preferred embodiment, it is provided that the wound care article comprises silver, silver ions or silver-containing compounds. A wound care article comprising silver and superabsorbent polymers is described in EP1656914, for example, which also describes the benefits resulting from the interplay of the two components. This interplay is enhanced in the context of the object of the present invention, since the higher conformability ensures a better contact with the wound, and the silver, silver ions or silver-containing compounds can therefore develop better their action in the wound. Also, the silver, the silver ions and the silver-containing compounds are mobilized more quickly due to the increased liquid absorption, so that they can enter the wound more easily. Furthermore, the foldability facilitates a use in deep cavities or fistulae, which are particularly susceptible to inflammation and therefore benefit particularly by treatment with silver, silver ions or silver-containing compounds.

In a further preferred embodiment it is provided that the wound care article comprises a preparation comprising bacteriophages or their components. A wound care article comprising bacteriophages or their components and superabsorbent polymers is described in WO 2009/060097, for example, which also describes the benefits resulting from the interaction of the two components. This interplay is enhanced in the context of the object of the present invention, since the higher conformability ensures a better contact with the wound, and the bacteriophages or their components can therefore develop better their action in the wound. Also, the bacteriophages or their components are mobilized more quickly due to the increased liquid absorption, so that they can enter the wound more easily. Furthermore, the foldability facilitates a use in deep cavities or fistulae, which are particularly susceptible to inflammation and therefore benefit particularly by treatment with bacteriophages or their components.

Further, it is provided the use of a wound care article according to any of the preceding claims as a tamponade, for the treatment of fistulae and/or treatment of wound cavities. For this purpose, the high conformability and/or foldability on the one hand, and the improved liquid absorption are particularly advantageous.

Furthermore, it is provided the use of a wound care article according to any of the preceding claims in a vacuum wound care system. Such vacuum wound care systems are described in the published patent applications WO2006/048240, WO2006/048246 and WO2006/056294, incorporated by reference in their entireties, which also describes the benefits resulting from the interplay of vacuum wound care and superabsorbent polymers.

This interplay is further enhanced in the context with the subject matter of the present invention as the higher conformability ensures a better contact with the wound, and the superabsorbent polymers can develop their retaining function better.

In a further preferred embodiment of the wound care article according to the invention at least one laminated layer is arranged between the absorption body and the envelope, preferably made from cellulosic nonwoven cellulose.

The laminated layer serves to direct the wound exudate absorbed by the envelope to the absorption body, and at the same time to prevent wound exudate from flowing back into the wound. The laminated layer is preferably adapted to the internal dimensions of the envelope and accordingly has a slightly larger area than the underlying absorption body, so that leakage from the absorption body can be contained. Preferably, the laminated layer is precisely adapted to the expansion space around the absorption body. The absorption body may be flanked by a laminated layer either only on one side, or covered by a sandwich structure from both sides with one or more laminated layers. Preferably, the laminated layer is made of cellulosic nonwovens without other superabsorbent components. However, laminated layers having superabsorbents are possible also.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A wound care article, comprising a planar absorption body made of a nonwoven material or air-laid material containing superabsorbent polymers, wherein said planar absorption body has a pattern of incisions and/or punchings.

2. The wound care article according to claim 1, characterized in that the incisions and/or punchings are formed and/or arranged in such a way so that they enhance the softness, conformability and/or the wearing comfort of the wound care article.

3. The wound care article according to claim 2, characterized in that the incisions and/or punchings are formed and/or arranged in such a way so that they facilitate the entry of liquids into the wound care article.

4. The wound care article according to claim 3, characterized in that the incisions and/or punchings are formed and/or arranged in such a way so that they define folding lines, along which the wound care article can be folded.

5. The wound care article according to claim 4, characterized in that it comprises an at least partially liquid-permeable envelope.

6. The wound care article according to claim 5, characterized in that the planar absorption body, in top view onto its planar side, has an area (F1) which in the non-wetted state is 3% to 75% smaller than the area (F2) of the flattened envelope.

7. The wound care article according to claim 6, characterized in that the absorption body is fixed in the envelope.

8. The wound care article according to claim 7, characterized in that the absorption body has a content of ≧40% by weight of superabsorbent polymers.

9. The wound care article according to claim 8, characterized in that the envelope comprises at least partially an elastic material.

10. The wound care article according to claim 9, characterized in that the wound care article comprises silver, silver ions or silver-containing compounds.

11. The wound care article according to claim 10, characterized in that the wound care article comprises a preparation comprising bacteriophages or their components.

12. A use of a wound care article according to claim 1 as tamponade for the treatment of fistulae and/or treatment of wound cavities.

13. A use of a wound care article according to claim 1 in a vacuum wound care system.
